(19) United States
(12) Patent Application Publication
(10) Pub. No.: US 2026/0224210 A1
(43) Pub. Date: Aug. 6, 2026

(54) MINIMALLY INVASIVE POSTERIOR APPROACH INSTRUMENTS

(71) Applicant: GLOBUS MEDICAL, INC., AUDUBON, PA (US)

(72) Inventors: Shawn Cox, Shrewsbury, PA (US); Noah Shephard, King of Prussia, PA (US)

(21) Appl. No.: 19/529,823

(22) Filed: Feb. 4, 2026

Related U.S. Application Data
(60) Provisional application No. 63/754,254, filed on Feb. 5, 2025.

(51) Int. Cl. A61B 17/02; A61B 17/00

(52) U.S. Cl. CPC A61B 17/0218; A61B 17/0206; A61B 2017/00477; A61B 2017/00876

(57) ABSTRACT

Provided are retractor blade systems comprising an arm adaptor and a substantially straight retractor blade. The arm adaptor is configured to removably couple to a retractor body, and the substantially straight retractor blade is configured to slideably and removeably couple to the arm adaptor via a connection mechanism. The connection mechanism is configured to hold the retractor blade in place during a surgical procedure while allowing the retractor blade to slide up and down when pushed or pulled by a surgeon.

### CROSS-REFERENCE TO RELATED APPLICATIONS

This application claims priority to U.S. Provisional Application No. 63/754,254, filed on February 5, 2025, the entire contents of which is incorporated herein.

### FIELD

The current disclosure is related to instruments for minimally invasive surgical procedures, and more specifically, instruments for use during minimally invasive posterior approach spine surgical procedures.

### BACKGROUND

Minimally invasive spine surgery (MISS) offers many benefits to patients including similar outcomes to open spine surgery while reducing operation time and post-op complications. The goals of minimally invasive approaches to the spine are to reduce surrounding tissue damage while maintaining optimal outcomes.

Wiltse (1964) described and popularized the intermuscular approach between the multifidus and erector spinae complex for posterior fusion. By approaching along the multifidus and lateral to the mammillary process to the level of the transverse process, this avoids the tendons and fixed neurovascular elements which are essential to the integrity of the back muscles. Using a microsurgical technique and an understanding of the periarticular anatomy facilitates preservation of this muscle integrity with segmental fixation and posterior fusion. Yet this approach is heavily constrained by these posterior segmental muscles, with specific regard to their neurovascular supply.

### SUMMARY

Provided herein are retractor blades configured to retract the surrounding muscles without damaging them, while still creating a large enough working window to treat the pathology. Current retractor blades and ports are unable to adequately fit between the posterior segmental muscles without causing damage. However, the retractor blades shown and described herein may be used in posterior lumbar interbody fusion (PLIF) and transforaminal lumbar interbody fusion (TLIF) procedures to reduce surrounding tissue damage, leading to better patient outcomes. To better insert the retractor blades without damaging the muscle, a handheld dissection tool (speculum) can be used that is capable of initially retracting the muscles. The retractor blades may then be inserted through the speculum to the desired position. Once in position, these retractor blades may attach and be compatible with existing retractor bodys (e.g., MARS 2V/3V). In order for these existing systems to hold the retractor blades, arm adapters may also be designed to attach to the existing system.

### BRIEF SUMMARY OF DRAWINGS

FIG. 1 shows a retractor system comprising arm adaptors holding retractor blades, according to some embodiments;

FIG. 2 shows a handheld dissection tool (e.g., speculum) configured to initially retract the muscle between the multifidus and erector spinae complex, according to some embodiments;

FIG. 3A shows a straight retractor blade for retracting the muscle between the multifidus and erector spinae complex, according to some embodiments;

FIG. 3B shows a straight retractor blade having an offset tip for retracting the muscle between the multifidus and erector spinae complex, according to some embodiments;

FIG. 4 depicts an adapter that attaches to an existing retractor body (e.g., MARS 2V/3V retractor body) while also holding onto the straight blade for muscle retraction, according to some embodiments;

FIG. 5 shows a variation of the spring-loaded plunger design shown in FIG. 4 by replacing the ball bearing with a roller wheel, according to some embodiments;

FIG. 6 shows a variation of the spring-loaded plunger design shown in FIG. 4 by replacing the ball bearing with a blunt narrow tip, according to some embodiments; and

FIG. 7 shows a quick-connect approach for the arm adapter using magnets that attach to the straight blade, according to some embodiments.

### DETAILED DESCRIPTION

Provided herein are retractor blades configured to retract surrounding muscles while causing little or no damage to the surrounding muscles. These retractor blades are able to retract the surrounding muscles with little to no damaged caused while at the same time providing a relatively large working window to treat the pathology.

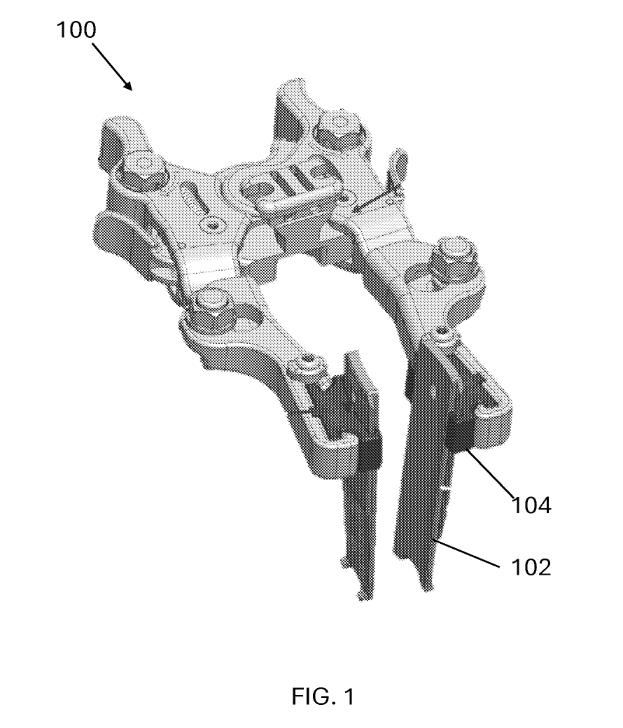

FIG. 1 shows an existing retractor system 100 comprising arm adaptors 104 holding retractor blades 102, according to some embodiments. As shown, the arm adaptors 104 connect the retractor blades 102 to the system 100. The system 100 shown in FIG. 1 represents a known device.

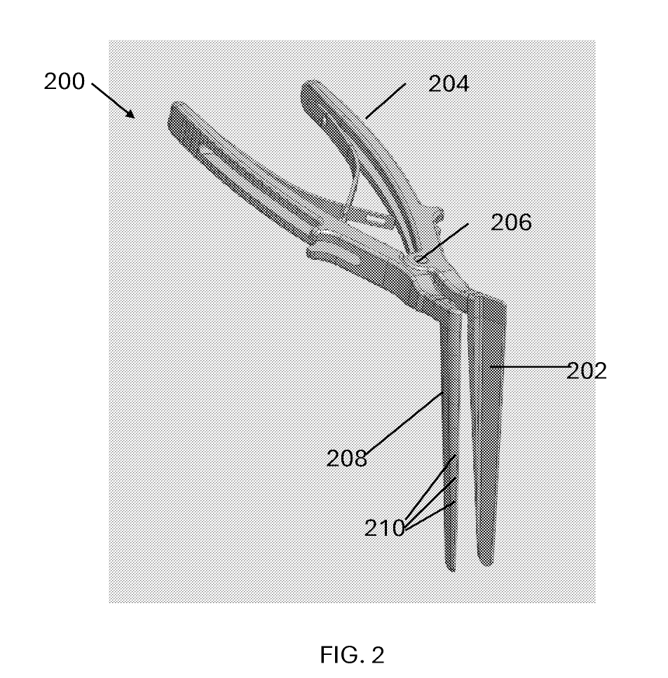

FIG. 2 shows a handheld dissection tool 200 (e.g., speculum) configured to retract muscle. In some embodiments, handheld dissection tool 200 is configured to retract muscle between the multifidus and erector spinae complex. Handheld dissection tool 200 may be configured to specifically perform an initial retraction. In embodiments in which handheld dissection tool 200 is configured to perform an initial retraction, this initial retraction creates room for retractor blades (e.g., retractor blades 3 of FIG. 1) to be dropped and guided down the speculum (i.e., hand dissection tool 200) next to the muscle.

The geometry of the inner surfaces 202 of the blades of the speculum (i.e., hand dissection tool 200) is designed to facilitate dropping retractor blades through the center and into the wound for a streamlined workflow. In some embodiments, an inner surface 202 of the hand dissection tool 200 comprises a planar surface. The inner surfaces 202 of each of the two blades 208 of hand dissection tool 200 may comprise planar surfaces that, when the hand dissection tool 200 is in a fully closed position, are parallel to each other. The inner surfaces 202 of each of the two blades 208 of hand dissection tool 200 may comprise planar surfaces that, when the hand dissection tool 200 is in a fully opened position, are parallel to each other.

In some embodiments, an inner surface 202 of one or both blades 208 may comprise a notch. For example, the notch may be located proximally (towards pivot 206). In some embodiments, the notch may be located distally (away from pivot 206). The notch may extend along the full length of each blade 208. In some embodiments, the notch may extend partially along the length of each blade 208. The notch may comprise a right angle. For example, the notch shown in FIG. 2 comprises a right angle. The size and shape of the inner surface 202 of the blades 208 may be designed to best allow retractor blades to drop into the retracted tissue opening.

The hand dissection tool 200 is in a fully closed position when in a resting state, when the handles 204 of the tool 200 are not in any way forced towards each other. By contrast, the hand dissection tool 200 is in a fully opened position when the two handles 204 of the tool 200 are forced together to a maximum state. In some embodiments, the handles 204 may be forced together by a user grip. Each handle 204 is integrally connected to a blade 208 of the tool 200. In some embodiments, a first handle 204 is integrally formed with a first blade 208 and a second handle 204 is integrally formed with a second blade 208. The first handle 204 and the first blade 208 are on the same side of a longitudinal axis, and the second handle 204 and the second blade 208 are on the opposite side of the longitudinal axis. The integrally-formed first handle 204 and first blade 208 are pivotably connected with the second handle 204 and the second blade 208 at pivot 206.

Once in place, the straight blades are then attached to the existing retractor system (e.g., MARS 2V/3V assembly) via bottom-loading into the retractor. The depth gauge markers 210 on the speculum show how far down the straight blades need to be dropped into the incision. These depth markers 210 correspond to the depth markers on the retractor blades shown and described herein, which allows the surgeon to reach the optimal depth to retract the muscle without causing damage.

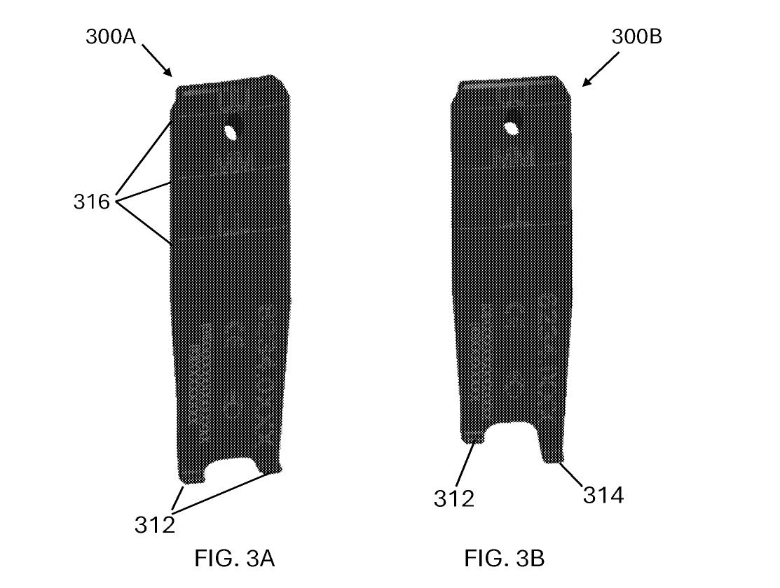

FIGS. 3A and 3B show straight retractor blades 300. Specifically, FIG. 3A shows a straight retractor blade 300A having equal tips 312, and FIG. 3B shows a straight retractor blade 300B having an offset tip 314. The offset tip 314 extends distally further than the other tip (tip 312) of retractor blade 300B.

In some embodiments, retractor blades 300 are specifically configured for retracting the muscle between the multifidus and erector spinae complex, according to some embodiments. Retractor blades 300 may be specifically designed for a posterior approach to a spinal procedure. The straight and flat profile of the retractor blades 300A and 300B creates a larger working window for the surgeon. In some embodiments, both an interior surface (i.e., the surface of the blade facing the working window) and an exterior surface (i.e., the surface adjacent patient tissue) are entirely planar. In some embodiments, both the interior surface (i.e., the surface of the blade facing the working window) and the exterior surface (i.e., the surface adjacent patient tissue) are substantially planar. The interior surface of retractor blade 300A and 300B may be planar except for the distal end of tip 312 and /or tip 314. In some embodiments, an interior surface of retractor blade 300A and/or 300B has a subtle curvature. In some embodiments, an exterior surface of retractor blade 300A and/or 300B has a subtle curvature.

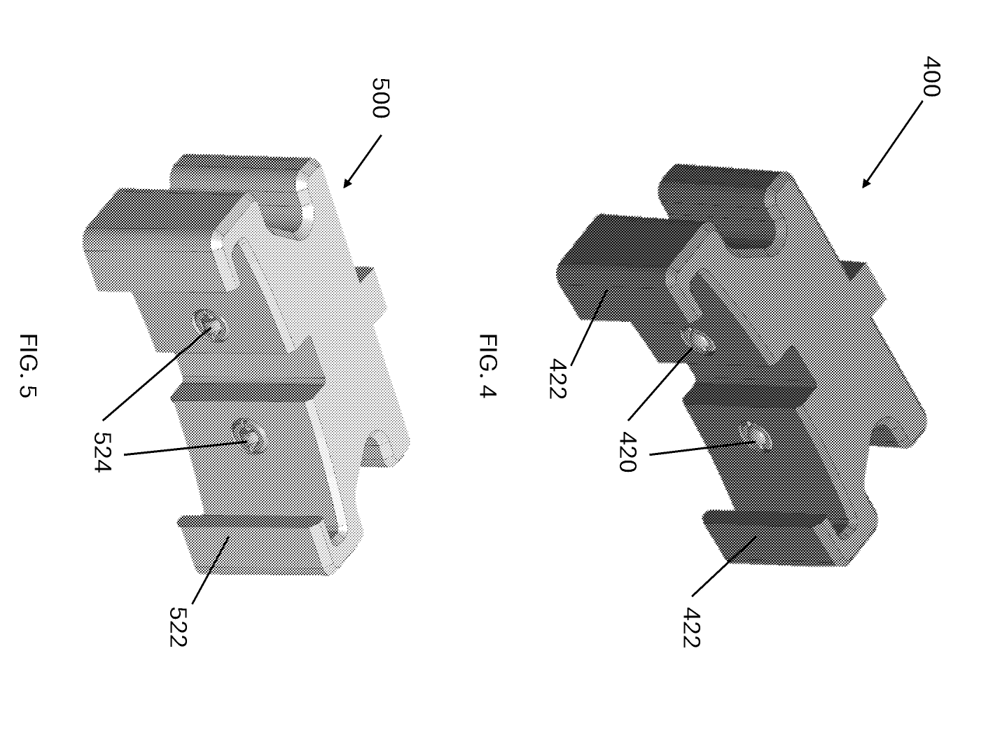

Depth gauge markers 316 on the side allow precise positioning of the blades into the incision based on depth markings read from the speculum (e.g., hand dissection tool 200). The narrow distal tip is 16mm wide and this small profile minimizes damage to the muscles when retracting. In some embodiments, the straight retractor blade 300B comprises an offset tip that allows the blades to rest on the transverse process (as shown in FIG. 3B). Offset tip end 314 allows the blade to more firmly dock to the bony anatomy of the patient. Blades 300A and 300B are also designed such that they can be loaded into arm adapters (e.g., arm adaptors shown and described with respect to any of FIG. 4-7) from either direction (i.e., top loading or bottom loading). In some embodiments, the two retractor blades 300A and 300B are interchangeable, meaning they can be coupled to either arm adaptor of the retractor system. In some embodiments, the two retractor blades 300A and 300B are directional, and may only be coupled to one of the two arm adaptors for the retractor system.

FIG. 4 depicts an arm adapter 400 that attaches to an existing retractor body (e.g., MARS 2V/3V retractor body, retractor system 100 of FIG. 1). The arm adaptor 400 is configured to couple a retractor blade (such as, for example, retractor blade 300A or 300B) to a retractor body. This allows the retractor body to rigidly hold in place the retractor blades during retraction.

In some embodiments, the arm adapter 400 can be locked in place to the retractor body. Arm adaptor 400 comprises two ball bearing spring loaded plungers 420 that are configured to apply a force to the inserted straight blade (e.g., blade 300A, 300B) to push it up against the curved arms 422 of the arm adaptor 400. This force creates friction between the straight retractor blade and a ball bearing spring loaded plunger 420 and between the curved arms 422 and the ball bearing spring loaded plungers 420. This friction can hold the straight blade in place during retraction of muscles, while still allowing it to adjustably slide up and down inside the arm adaptor when pulled or pushed by the surgeon. The open design on the bottom and top faces allows for the straight blades to be top or bottom-loaded depending on surgeon preference. Arm adapters 400 can come in a “left” and “right” version. In some embodiments, arm adaptors 400 are interchangeable and not sided (i.e., they are not “left” or “right”).

In some embodiments, arm adaptor 400 may comprise only one ball bearing spring loaded plunger 420. In some embodiments, arm adaptor 400 may comprise 2, 3, 4, 5, or 6 ball bearing spring loaded plungers 420. Arm adaptor 400 may comprise two ball bearing spring loaded plungers 420, as is shown in FIG. 4.

FIG. 5 shows arm adaptor 500 having a different variation of the spring-loaded plunger design shown in FIG. 4. Here, instead of the ball bearing spring loaded plunger, arm adaptor 500 comprises two roller wheels 524. Roller wheels 524 operate similarly to that of the ball bearing spring loaded plungers 420 in that the roller wheels are configured to apply a force to the inserted straight blade (e.g., blade 300A, 300B) to push it up against the curved arms 522 of the arm adaptor 500. This force creates friction between the straight retractor blade and roller wheels 524 as well as between the curved arms 522 and the roller wheels 524. This friction can hold the straight blade in place during retraction of muscles, while still allowing it to adjustably slide up and down inside the arm adaptor 500 when pulled or pushed by the surgeon. The open design on the bottom and top faces of arm adaptor 500 allows for the straight blades to be top or bottom-loaded depending on surgeon preference.

Like arm adaptor 400, arm adaptor 500 can come in a “left” and “right” version or it may be non-directional. In some embodiments, arm adaptor 500 may comprise only one roller wheel 524. In some embodiments, arm adaptor 500 may comprise 2, 3, 4, 5, or 6 roller wheels 524. Arm adaptor 500 may comprise two roller wheels 524, as is shown in FIG. 5.

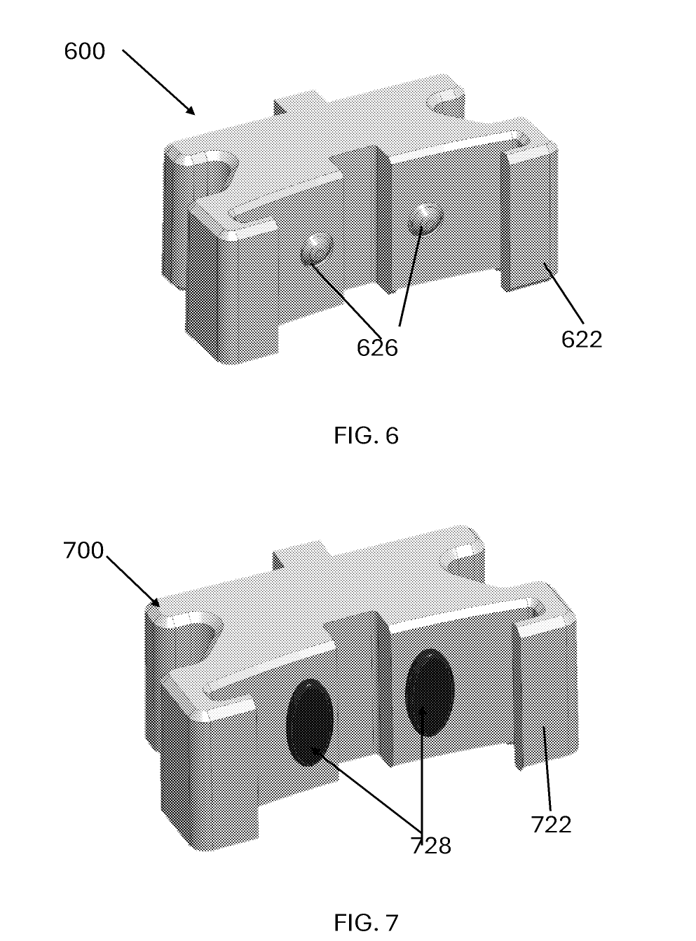

FIG. 6 shows an arm adaptor 600 having another variation of the spring-loaded plunger design shown in FIG. 4. Specifically, the arm adaptor 600 of FIG. 6 comprises a blunt narrow tip 626 instead of the ball bearing spring loaded plunger 420 of arm adaptor 400 or the roller wheel 524 of arm adaptor 500. Blunt narrow tip 626 operates similarly to that of the ball bearing spring loaded plungers 420 and roller wheels 524 in that the blunt narrow tips 626 are configured to apply a force to the inserted straight blade (e.g., blade 300A, 300B) to push it up against the curved arms 622 of the arm adaptor 600. This force creates friction between the straight retractor blade and blunt narrow tip 626 as well as between the curved arms 622 and the blunt narrow tip 626. This friction can hold the straight blade in place during retraction of muscles, while still allowing it to adjustably slide up and down inside the arm adaptor 600 when pulled or pushed by the surgeon. The open design on the bottom and top faces of arm adaptor 600 allows for the straight blades to be top or bottom-loaded depending on surgeon preference.

Like arm adaptors 400 and 500, arm adaptor 600 can come in a “left” and “right” version or it may be non-directional. In some embodiments, arm adaptor 600 may comprise only one blunt narrow tip 626. In some embodiments, arm adaptor 600 may comprise 2, 3, 4, 5, or 6 blunt narrow tips 626. Arm adaptor 600 may comprise two blunt narrow tips 626, as is shown in FIG. 6.

FIG. 7 shows an arm adaptor 700 comprising a quick-connect approach for the arm adapter using magnets 728 that attach to the straight blade. This is a different variation to that of arm adaptors 400 (ball bearing spring loaded plunger), 500 (roller wheel), and 600 (blunt narrow tip). Magnets 728 operate similarly to that of the other connection mechanisms shown and described in FIGS. 4-6 in that the magnets 728 are configured to apply a force to the inserted straight blade (e.g., blade 300A, 300B) to push it up against the curved arms 722 of the arm adaptor 700. This force creates friction between the straight retractor blade and magnets 728 as well as between the curved arms 722 and the magnets 728. This friction can hold the straight blade in place during retraction of muscles, while still allowing it to adjustably slide up and down inside the arm adaptor 700 when pulled or pushed by the surgeon. The open design on the bottom and top faces of arm adaptor 700 allows for the straight blades to be top or bottom-loaded depending on surgeon preference.

Like arm adaptor 400, 500, and 600, arm adaptor 700 can come in a “left” and “right” version or it may be non-directional. In some embodiments, arm adaptor 700 may comprise only one magnet 728. In some embodiments, arm adaptor 700 may comprise 2, 3, 4, 5, or 6 magnets 728. Arm adaptor 700 may comprise two magnets 728, as is shown in FIG. 7.

The advantage of the arm adaptors shown and described herein are their ability to allow the straight blade to move up and down when adjusted, while still firmly holding it in position during retraction. This allows the surgeon to custom adjust the retraction position in order to best avoid damaging the surrounding muscle tissue of the patient.

The straight blades shown and described herein have a flat and narrow profile which help increase the working window for the surgeon while preserving muscle integrity. Other blades and ports are curved, and therefore more difficult to insert without damaging muscle. The curvature of existing retractor blades also limits the working window.

Additional aspects, advantages and/or other features of example embodiments of the invention will become apparent in view of this detailed description. It should be apparent to those skilled in the art that the described embodiments provided herein are merely exemplary and illustrative and not limiting. Numerous embodiments and modifications thereof are contemplated as falling within the scope of this disclosure and equivalents thereto.

Further, although the invention has been described in detail and with reference to specific embodiments, it will be apparent to one skilled in the art that various changes and modifications can be made without departing from the spirit and scope of the invention. Thus, it is intended that the invention covers the modifications and variations of this invention provided they come within the scope of the appended claims and their equivalents. It is expressly intended, for example, that all components of the various devices disclosed above may be combined or modified in any suitable configuration.

1. A retractor system comprising:
a first arm adaptor configured to removably couple to a retractor body; and
a first retractor blade configured to slideably and removeably couple to the first arm adaptor via a first connection mechanism, wherein the first connection mechanism is configured to hold the first retractor blade in place during a surgical procedure while allowing the first retractor blade to slide up and down when pushed or pulled by a surgeon.

2. The retractor system of claim 1, wherein the first arm adaptor comprises a pair of curved arms configured to wrap around opposing side edges of the first retractor blade when the first retractor blade is coupled to the first arm adaptor.

3. The retractor system of claim 1, comprising a second arm adaptor configured to removably couple to the retractor body, wherein the first arm adaptor is a left arm adaptor and the second arm adaptor is a right arm adaptor.

4. The retractor system of claim 3, comprising a second retractor blade configured to slideably and removeably couple to the second arm adaptor via a second connection mechanism, wherein the second connection mechanism is configured to hold the second retractor blade in place during a surgical procedure while allowing the second retractor blade to slide up and down when pushed or pulled by the surgeon.

5. The retractor system of claim 1, wherein the first arm adaptor comprises an open upper face and an open lower face.

6. The retractor system of claim 5, wherein the first retractor blade is configured to be loaded onto the first arm adaptor from either the lower face or the upper face.

7. The retractor system of claim 1, wherein the first arm adaptor comprises only one first connection mechanism.

8. The retractor system of claim 1, wherein the first arm adaptor comprises exactly two first connection mechanisms.

9. The retractor system of claim 1, wherein the first connection mechanism comprises a ball bearing spring loaded plunger.

10. The retractor system of claim 1, wherein the first connection mechanism comprises a roller wheel.

11. The retractor system of claim 1, wherein the first connection mechanism comprises a blunt narrow tip.

12. The retractor system of claim 1, wherein the first connection mechanism comprises a magnet.
